(19) United States
(12) Patent Application Publication
(10) Pub. No.: US 2024/0286107 A1
(43) Pub. Date: Aug. 29, 2024

(54) CATALYTIC NON-THERMAL PLASMA ASSISTED CONVERSION APPARATUS AND METHOD

(71) Applicants: SUSTEON INC., Cary, NC (US); SOUTHERN CALIFORNIA GAS COMPANY, Los Angeles, CA (US)

(72) Inventors: Shaojun James Zhou, Cary, NC (US); Raghubir Prasad Gupta, Durham, NC (US)

(21) Appl. No.: 18/693,360

(22) PCT Filed: Sep. 21, 2022
(86) PCT No.: PCT/US2022/076756

§ 371 Date: Mar. 19, 2024

Related U.S. Application Data
(60) Provisional application No. 63/246,338, filed on Sep. 21, 2021.

(51) Int. Cl. B01J 19/08; B01J 8/04; B01J 8/06; C01B 3/26; C01B 3/38; C01B 32/05; C01C 1/04; C07C 2/76; C07C 5/48; C07C 29/48

(52) U.S. Cl. CPC B01J 19/088; B01J 8/0465; B01J 8/0492; B01J 8/065; C01B 3/26; C01B 3/38; C01B 32/05; C01C 1/0494; C07C 2/76; C07C 5/48; C07C 29/48; B01J 2219/083; B01J 2219/0875; B01J 2219/0892; B01J 2219/0896; C01B 2203/0233; C01B 2203/0238; C01B 2203/1017; C01B 2203/1223; C01B 2203/1241

(57) ABSTRACT

A dielectric barrier discharge plasma reactor and method in which plasma is used to activate difficult-to-activate molecules and the catalyst so that chemical conversion of the activated molecules can occur at reduced temperature and pressure conditions to carry out chemical reactions that ordinarily occur at high temperature and high pressure conditions or otherwise do not occur at all. The dielectric barrier discharge plasma reactor includes a tubular outer ground electrode having an inner surface bounding an interior volume therein, a dielectric electrode coaxially mounted in the interior volume of the tubular outer ground electrode, the dielectric electrode comprising a central electrode in a cylindrical dielectric element, the cylindrical dielectric element having an outer surface in spaced relationship to the inner surface of the tubular outer ground electrode to define an annular fluid flow passage therebetween, and a catalyst material comprising catalyst coated on the inner surface of the tubular outer ground electrode and optionally further comprising catalyst in a catalyst bed in the annular fluid flow passage.

### CROSS-REFERENCE TO RELATED APPLICATION

The benefit under 35 USC § 119 of U.S. Provisional Patent Application 63/246,338 filed Sep. 21, 2021 in the names of Shaojun James Zhou and Raghubir Prasad Gupta for “CATALYTIC NON-THERMAL PLASMA-ASSISTED CONVERSION APPARATUS AND METHOD” is hereby claimed. The disclosure of U.S. Provisional Patent Application 63/246,338 is hereby incorporated herein by reference, in its entirety, for all purposes.

### FIELD

The present disclosure generally relates to a catalytic non-thermal plasma assisted conversion apparatus and method. More specifically, the disclosure relates to a dielectric barrier discharge plasma reactor and method in which plasma is used to activate difficult-to-activate molecules and the catalyst so that chemical conversion of the activated molecules can occur at reduced temperature and pressure conditions to carry out chemical reactions that ordinarily occur at high temperature and high pressure conditions or otherwise do not occur at all.

### DESCRIPTION OF THE RELATED ART

In a large number of industrial chemical processes, reactions are employed that require conditions of high temperature and high pressure in order to be carried out. Examples include methane steam reforming, methanol steam reforming, methane dry reforming, ammonia production, ethane oxidative dehydrogenation, conversion of methane to aromatics, conversion of methane to methanol, production of syngas from natural gas, conversion of CO₂ to useful chemical products, and conversion of methane to carbon and hydrogen.

Such reactions when carried out at conditions of high temperature and high pressure entail high capital equipment and operating costs that impact their economic character and applicability.

Other reactions that are conceptually possible nonetheless involve reactant species that are so difficult to activate that considerations of enthalpy and free energy preclude their successful commercial usage.

In consequence, the art continues to seek improvements in ameliorating high temperature and high pressure requirements for reactions of the foregoing types and in facilitating reactions involving difficult-to-activate potential reactant species.

### SUMMARY

The present disclosure relates to dielectric barrier discharge plasma reactor apparatus and method for conducting chemical reactions at favorable temperature and pressure reaction conditions.

In one aspect, the disclosure relates to a dielectric barrier discharge plasma reactor that includes a tubular outer ground electrode having an inner surface bounding an interior volume therein, a dielectric electrode coaxially mounted in the interior volume of the tubular outer ground electrode, the dielectric electrode comprising a central electrode in a cylindrical dielectric element, the cylindrical dielectric element having an outer surface in spaced relationship to the inner surface of the tubular outer ground electrode to define an annular fluid flow passage therebetween, and a catalyst material comprising catalyst coated on the inner surface of the tubular outer ground electrode and optionally further comprising catalyst in a catalyst bed in the annular fluid flow passage.

In another aspect, the disclosure relates to a multi-tube dielectric barrier discharge plasma reactor, comprising multiple ones of the dielectric barrier discharge plasma reactor of the present disclosure, arranged for concurrent passage of fluid therethrough.

In a further aspect, the disclosure relates to a method of reacting fluid reactants to form reaction product(s), comprising: flowing the fluid reactants through an annular flow passage bounded by an outer tubular ground electrode and an inner dielectric electrode in the presence of catalyst material comprising catalyst coated on the inner surface of the tubular outer ground electrode optionally further comprising catalyst in a catalyst bed in the annular fluid flow passage; energizing the inner dielectric electrode to generate a dielectric barrier discharge plasma of the fluid reactants in the annular flow passage inducing reaction of the fluid reactants to form the reaction product(s); and discharging the reaction products from the annular flow passage.

A further aspect of the disclosure relates to the method described above, wherein the flowing, energizing, and discharging are conducted in multiple ones of the annular flow passage, in a multi-tube dielectric barrier discharge plasma reactor.

Other aspects, features and embodiments of the disclosure will be more fully apparent from the ensuing description and appended claims.

### BRIEF DESCRIPTION OF THE DRAWINGS

FIG. 1 is a cross-sectional side view of a dielectric barrier discharge plasma reactor, according to one embodiment of the present disclosure.

FIG. 2 is a transverse cross-sectional view of the dielectric barrier discharge plasma reactor of FIG. 1, showing the dimensional characteristics thereof.

FIG. 3 is a transverse cross-sectional view of a dielectric barrier discharge plasma reactor, according to another embodiment of the disclosure.

FIG. 4 is a schematic perspective view of a dielectric barrier discharge plasma reactor according to yet another embodiment of the disclosure.

FIG. 5 is a transverse cross-sectional view of a multi-tube reactor according to one embodiment of the present disclosure, containing an array of tubular reactors constructed in accordance with the present disclosure.

### DETAILED DESCRIPTION

The present disclosure relates to catalytic non-thermal plasma assisted conversion apparatus and method, for effecting chemical reactions at advantageous reaction conditions, and that have particular utility for carrying out chemical reactions involving hard-to-activate activatable reactant species.

In one aspect, the disclosure relates to a dielectric barrier discharge plasma reactor that includes a tubular outer ground electrode having an inner surface bounding an interior volume therein, a dielectric electrode coaxially mounted in the interior volume of the tubular outer ground electrode, the dielectric electrode comprising a central electrode in a cylindrical dielectric element, the cylindrical dielectric element having an outer surface in spaced relationship to the inner surface of the tubular outer ground electrode to define an annular fluid flow passage therebetween, and a catalyst material comprising catalyst coated on the inner surface of the tubular outer ground electrode and optionally further comprising catalyst in a catalyst bed in the annular fluid flow passage.

In such dielectric barrier discharge plasma reactor, the cylindrical dielectric element may comprise at least one dielectric material of glass, ceramic, dielectric polymers, and dielectric metal oxides, or any other suitable material. In one embodiment of the disclosure, the cylindrical dielectric element comprises quartz glass.

The dielectric barrier discharge plasma reactor in various embodiments may be constructed with the cylindrical dielectric element of the dielectric electrode comprising a tube, in which the central electrode is disposed. In other embodiments, the cylindrical dielectric element of the dielectric electrode comprises a dielectric enamel on the central electrode.

In another aspect, the disclosure relates to a multi-tube dielectric barrier discharge plasma reactor, comprising multiple ones of the dielectric barrier discharge plasma reactor as variously described herein, arranged for concurrent passage of fluid therethrough. In such multi-tube dielectric barrier discharge plasma reactor, the multiple ones of the dielectric barrier discharge plasma reactor may be mounted in a shell. The multi-tube dielectric barrier discharge plasma reactor may be constructed and arranged, with the multiple ones of the dielectric barrier discharge plasma reactor being parallelly aligned with one another in the shell.

Another aspect of the disclosure relates to a method of reacting fluid reactants to form reaction product(s), comprising: flowing the fluid reactants through an annular flow passage bounded by an outer tubular ground electrode and an inner dielectric electrode in the presence of catalyst material comprising catalyst coated on the inner surface of the tubular outer ground electrode optionally further comprising catalyst in a catalyst bed in the annular fluid flow passage; energizing the inner dielectric electrode to generate a dielectric barrier discharge plasma of the fluid reactants in the annular flow passage inducing reaction of the fluid reactants to form the reaction product(s); and discharging the reaction products from the annular flow passage.

The reaction conducted in such method may be of any appropriate type, and in specific embodiments may comprise: methane steam reforming; methanol steam reforming; methane dry reforming; ammonia production, ethane oxidative dehydrogenation; conversion of methane to aromatics; conversion of methane to methanol; production of syngas from natural gas; or conversion of methane to carbon and hydrogen. In various embodiments, the fluid reactants may comprise methane, or carbon dioxide, or nitrogen. The energizing of the inner dielectric electrode in such method may be carried out at any suitable voltage. In various embodiments, such voltage may be at least 10 kV. In other embodiments, the energizing of the inner dielectric electrode imposes a voltage in a range of from 10 kV to 50 kV.

A further aspect of the disclosure relates to the method described above, wherein the flowing, energizing, and discharging are conducted in multiple ones of the annular flow passage, in a multi-tube dielectric barrier discharge plasma reactor.

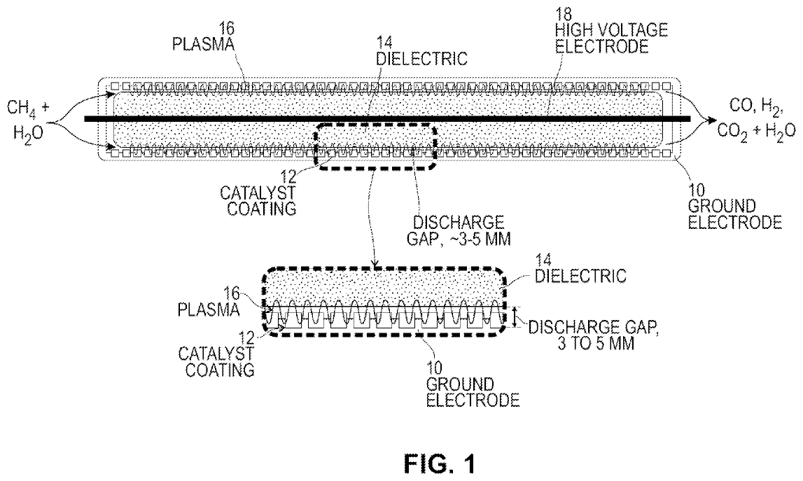

Referring now to the drawings, FIG. 1 is a cross-sectional side view of a dielectric barrier discharge plasma reactor, according to one embodiment of the present disclosure. The reactor is illustratively depicted in a methane steam reforming operation in which methane and water are flowed into the reactor at the reactor inlet for reaction in the reactor, to form carbon monoxide, hydrogen, and carbon dioxide as reaction products that are discharged from the reactor at the outlet thereof, with residual water.

The reactor thus is of elongate character, including an outer tubular ground electrode 10, the interior surface of which, as shown in the enlarged inset view of a portion of the reactor structure, has a catalyst coating 12 thereon. The catalyst may be of any suitable type, and may for example comprise transition metals, mixed metal oxides, or other catalytically active elements, compounds, and combinations, including catalyst compositions with dopant species and/or promoters, etc. The catalyst composition may include a support, which can be of any suitable type, and may for example comprise silica, alumina, a macroreticulate resin material, etc. In a specific embodiment, the catalyst may comprise nickel/alumina. The catalyst coating may be of any suitable thickness, and may be continuous or discontinuous in character, but preferably is of a continuous or substantially continuous character.

The reactor further includes a central dielectric element 14, which may be of cylindrical form, with a high-voltage central electrode 18 embedded in and extending through the dielectric element, as shown. In such arrangement, a discharge gap is formed between the outer tubular ground electrode 10 and the dielectric element 14, which may for example be on the order of about 3-5 mm in radial distance between the catalyst-coated interior surface of the outer tubular ground electrode, and the outer surface of the dielectric element.

When the high-voltage central electrode 18 is energized at suitable voltage, a plasma 16 is formed of the reactant gases being flowed through the discharge gap so that such gases are energetically activated to facilitate their reaction to produce the desired reaction products.

As used herein, the term “high-voltage” in reference to the central electrode in the cylindrical dielectric element means a voltage in a range of from 10 kV to 50 kV.

Although the dielectric barrier discharge plasma reactor shown in FIG. 1 has been illustratively described as bearing a catalyst coating on the interior surface of the outer tubular ground electrode, the reactor may alternatively or additionally contain catalyst in suitable particulate form in the discharge gap. In such arrangement, the particulate catalyst may form an annular catalyst bed in the discharge gap. As a still further variant, the discharge gap may contain support material of appropriate character and composition, on which catalyst is supported. It will be appreciated that the specific arrangement of particulate catalyst and/or supported catalyst will entail consideration and selection of size, shape, and packing characteristics of the catalyst and/or supported catalyst, providing appropriate amount of catalyst and catalyst contact area for interaction with the reactant gases, with appropriate pressure drop, flow conductance, and hydrodynamic character being provided.

The dielectric element in the dielectric barrier discharge plasma reactor may be of any appropriate character, and may for example comprise glass, ceramic, dielectric polymers, dielectric metal oxides, etc.

In the reactor of the present disclosure, each of the dielectric electrode, and its constituent high-voltage central electrode and cylindrical dielectric element, is imperforate along its extent (length) in the reactor, and devoid of any cage or Faraday structures for retention of catalyst. The reaction chamber in the reactor of the present disclosure is a single reaction chamber constituted by the annular volume between the dielectric electrode and the outer ground electrode, wherein the outer ground electrode may have a catalyst coating thereon.

At its respective ends, the central electrode of the dielectric electrode may be mounted in an insulative bushing or other insulative structure isolating such electrode from the outer ground electrode, and the central electrode may be joined by appropriate electrical circuitry to a voltage generator, power supply, or the like, to provide the central electrode with appropriate voltage to generate a non-thermal plasma of the fluid reactants in the reactor annular chamber.

The reactor of the present disclosure may be constructed so that the reactor annular chamber at its respective ends communicates with fluid flow passages for introducing fluid reactants at an inlet end and discharging reaction product(s) at an outlet end, and such fluid flow passages may be provided in respective header or manifold structures, with the inlet end fluid passages coupled in fluid flow relationship with a source or sources of the fluid reactants, and with the outlet end fluid passages coupled in fluid flow relationship with a collection structure, such as a gas storage and dispensing vessel, tube trailer, or a pipeline or other fluid flow circuitry that is operative to transmit the fluid product(s) to a further processing apparatus, point of use, or other disposition.

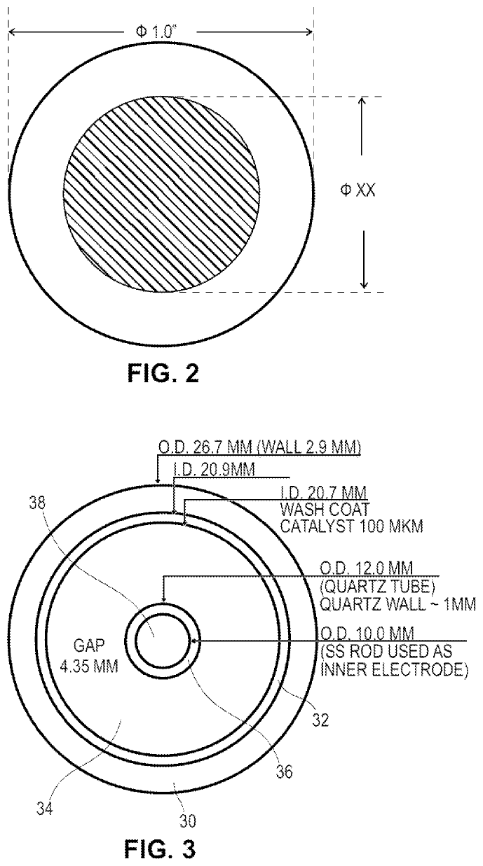

FIG. 2 is a simplified transverse cross-sectional view of the dielectric barrier discharge plasma reactor of FIG. 1, showing the dimensional characteristics thereof. In the illustrative drawing, the outer diameter of the tubular ground electrode is 1 inch (2.54 cm), and the outer diameter of the cylindrical dielectric electrode (cylindrical dielectric element containing the high-voltage central electrode embedded therein, as previously described), as coaxially disposed in the tubular ground electrode interior volume, is shown as dimension “xx” which may for example be 0.5 inch (1.27 cm).

FIG. 3 is a transverse cross-sectional view of a dielectric barrier discharge plasma reactor, according to another embodiment of the disclosure, showing illustrative dimensions thereof. The reactor as shown in FIG. 3 includes an outer electrode tube 30 on the interior surface of which is disposed a catalyst coating 32. Coaxially located in the interior volume of the catalyst-coated outer electrode tube is a quartz tube 36 containing a stainless steel rod 38 as an inner electrode. Such arrangement provides an annular gap 34, between the catalyst-coated outer electrode tube interior surface, and the outer surface of the quartz tube 36, through which reactant gases are flowed in the operation of the reactor.

In the illustrative dimensions set out in FIG. 3, the outer electrode tube 30 has an outer diameter of 26.7 mm (1.05 inch), and an inner diameter of 20.9 mm (0.824 inch), with the wash coat of catalyst on the inner surface of such tube having a thickness of 100 μm (0.0039 inch), so that the inner diameter of the catalyst-coated outer electrode tube is 20.7 mm (0.815 inch). The wall thickness of the outer electrode tube is 2.9 mm (0.114 inch). The quartz tube 36 in this illustrative embodiment has an outer diameter of 12 mm (0.472 inch), and a wall thickness of 1 mm (0.039 inch), with the stainless steel rod 38 in the quartz tube having an outer diameter of 10 mm (0.394 inch). A gap of 4.35 mm (0.171 inch) radial distance between the catalyst-coated outer electrode tube interior surface and the outer surface of the quartz tube is thereby provided.

In the general practice of the present disclosure, the gap between electrodes can be varied for plasma generation, catalyst loading, and gas space velocity, as can be determined on the basis of the present disclosure, by persons of ordinary skill in the art. Voltage and frequency can be varied for plasma generation and control of plasma stability.

FIG. 3 thus depicts a quartz tube being utilized as a dielectric element, inserted over the stainless steel rod serving as the inner electrode of the reactor. Alternatively, such inner electrode rod could be coated with a dielectric enamel coating, to provide the cylindrical dielectric electrode for the reactor.

The view shown in FIG. 3 is a transverse cross-section of the tubular reactor, and it will be appreciated that the length of the reactor may be of any suitable character consistent with the requirements of the reaction system in which the tubular reactor is deployed. By way of specific example, the outer electrode tube may be a tube formed of 316 stainless steel, having a length of 103.5 cm (40.75 inch). As previously noted, a packed bed of particulate catalyst may be deployed in the annular gap of the reactor, as an alternative to the catalyst coating on the inner surface of the outer electrode tube, or in addition to such catalyst coating on the inner surface of the outer electrode tube.

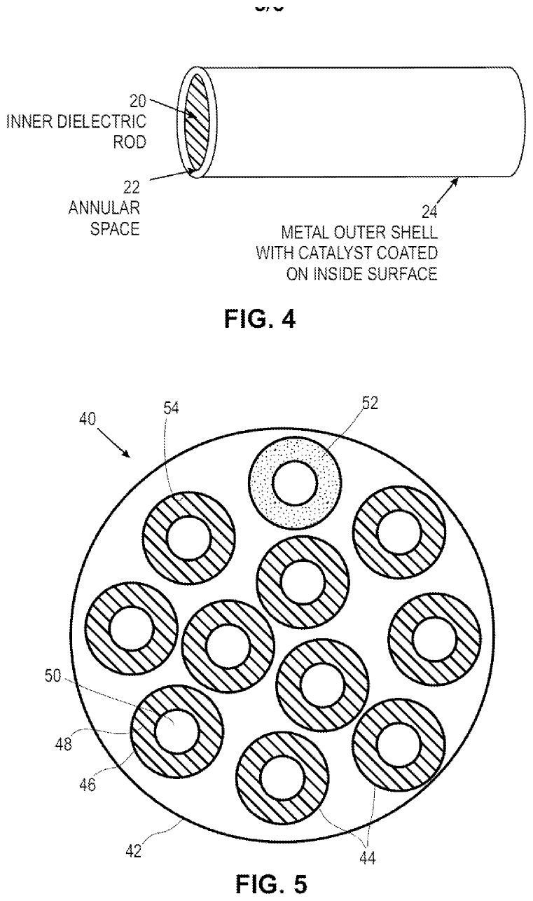

FIG. 4 is a simplified schematic perspective view of a dielectric barrier discharge plasma reactor according to yet another embodiment of the disclosure, comprising a metal outer shell 24 which catalyst coated on the inside surface thereof, with an inner dielectric rod 20, and annular space 22 therebetween. The length and other dimensional characteristics of the reactor, as well as the specific materials of construction of the reactor components, may be widely varied in the general practice of the present disclosure.

FIG. 4 thus shows a single tube reactor that may be utilized in carrying out reactions of widely varied character during generation of plasma in the annular space 22 when the inner dielectric rod is energized at appropriate voltage. A multiplicity of such tubes can be employed to constitute a multi-tube reactor assembly, as mounted in a reactor shell or housing, and arranged with the inner electrode of each constituent tubular reactor being concurrently energized for processing of reactant gases. The outer ground electrodes in such arrangement may be commonly grounded with respect to one another, via the reactor shell or housing, or in other suitable manner.

FIG. 5 is a transverse cross-sectional view of a multi-tube reactor 40 according to one embodiment of the present disclosure, containing an array of tubular reactors 44 constructed in accordance with the present disclosure, mounted in reactor shell 42. Each of the tubular reactors 44 includes a ground electrode 46 and a dielectric electrode 50 forming a gap 48 therebetween. The tubular reactors in the array may include an annular catalyst 52 in the gap 48, and/or a catalyst coating 54 on an interior surface of the ground electrode 46.

In an illustrative steam reforming operation, a dielectric barrier discharge plasma reactor in accordance with the present disclosure was operated for steam reforming of methane with the operational characteristics and performance results set out in Table 1 below.

TABLE 1
Steam Reforming of Methane to Produce Hydrogen
Operational Characteristics/Performance Results

| Reaction temperature | 450° C. | |
|---|---|---|
| Voltage | 12 | kV |
| Plasma power | 85 | W |
| Power consumption (in situ | 170 | W |
| steam generation included) | | |
| CH₄ inlet flow rate | 400 | cm³/minute |
| CH₄ molar flow rate | 0.01785 | mole per minute |
| Catalyst volume | 12 | cm³ |
| Residence time | 1.8 | seconds |
| CH₄ space velocity | 1.10 | cm/second |
| GHSV (STP) | 2000 | hr⁻¹ |
| Steam/carbon ratio | 3 | |
| CH₄ conversion | 90% | |
| H₂ production rate | 138.8 | grams/day |
| Reactor length | 1.97 | cm |
| Reactor cross-sectional area | 6.08 | cm² |
| Steam rate | 0.05354 | mole per minute |
| Power consumption for | 43 | W |
| steam generation | | |
| Heated vaporization of H₂O | 40.65 | kJ/mole |
| Specific heat (cP) of H₂O | 75.3 | J/K/mole |

Set out in Table 2 below is an illustrative tabulation of reactor design variables for single-tube reactor and multi-tube reactor systems in accordance with the present disclosure.

TABLE 2

| Design | | System | System | System | System | System | System | System |
|---|---|---|---|---|---|---|---|---|
| variable | Units | 1 | 2 | 3 | 4 | 5 | 6 | 7 |
| H₂ | kg/day | 2.00 | 4.00 | 25.00 | 50.00 | 100.00 | 200.00 | 300.00 |
| production | | | | | | | | |
| rate | | | | | | | | |
| Shell | cm | 2.54 | 2.54 | 2.54 | 2.54 | 2.54 | 2.54 | 2.54 |
| diameter | | | | | | | | |
| Electrode | cm | 1.27 | 1.27 | 1.27 | 1.27 | 1.27 | 1.27 | 1.27 |
| diameter | | | | | | | | |
| Space | hr⁻¹ | 2000 | 2000 | 2000 | 2000 | 2000 | 2000 | 2000 |
| velocity | | | | | | | | |
| Lab scale | grams/ | 138.4 | 138.4 | 138.4 | 138.4 | 138.4 | 138.4 | 138.4 |
| unit H₂ | day | | | | | | | |
| rate | | | | | | | | |
| Scale up | | 14.45 | 28.90 | 180.6 | 361.2 | 722.5 | 1445 | 2167 |
| factor | | | | | | | | |
| CH₄ flow | liters/ | 5.78 | 11.56 | 72.25 | 144.5 | 289.0 | 578.0 | 867.0 |
| rate | minute | | | | | | | |
| Catalyst | cm³ | 173.4 | 346.8 | 2167 | 4335 | 8670 | 17339 | 26009 |
| volume | | | | | | | | |
| Length of | cm | 45.63 | 91.25 | 570.3 | 1141 | 2281 | 4563 | 6844 |
| reactor | | | | | | | | |
| Gas | cm/ | 1521 | 3042 | 19011 | 38022 | 76044 | 152088 | 228132 |
| velocity | minute | | | | | | | |
| Residence | Seconds | 1.80 | 1.80 | 1.80 | 1.80 | 1.80 | 1.80 | 1.80 |
| time | | | | | | | | |
| Number of | | 1 | 2 | 12 | 24 | 48 | 96 | 72 |
| parallel | | | | | | | | |
| tubes | | | | | | | | |
| Length of | cm | 45.63 | 45.63 | 47.53 | 47.53 | 47.53 | 47.53 | 95.06 |
| parallel | | | | | | | | |
| tubes | | | | | | | | |
| Estimated | Kilowatts | 1.23 | 2.46 | 15.4 | 30.7 | 61.4 | 122.8 | 184.2 |
| plasma | | | | | | | | |
| power | | | | | | | | |

The features and advantages of the disclosure are further illustrated with reference to the following examples, which are not to be construed as in any way as limiting the scope of the disclosure but rather as illustrative of respective embodiments thereof, in particular implementations.

### Example 1

### Methane Steam Reforming (SMR)

In the SMR reaction, natural gas, comprised mainly of methane, CH₄, is reacted with steam according to the following equations (1) and (2), to produce carbon monoxide and hydrogen.

$$\begin{matrix}{CH}_{4} + {H}_{2} O ⇌ CO + 3 {H}_{2} & Δ {H}_{r} = 206 kJ / mol\end{matrix} \qquad ( 1 )$$

Additional hydrogen is produced at reaction temperatures by the water-gas shift reaction:

$$\begin{matrix}CO + {H}_{2} O ⇌ {CO}_{2} + {H}_{2} & {ΔH}_{r} = - 41 kJ / mol\end{matrix} , \qquad ( 2 )$$

where ΔHᵣ is the standard enthalpy of reaction. In typical conventional commercial operations, SMR produces 10 metric tons of CO₂ per metric ton of H₂, of which 17 to 41% is the direct product of hydrocarbon combustion to provide the heat required for carrying out the chemical reaction at 700° C. to 1,000° C.

The foregoing SMR reaction can be carried out in a dielectric barrier discharge plasma reactor constructed and operated in accordance with the present disclosure, at temperatures below 500° C. with high methane conversion and high hydrogen production because reaction (2) is favored at lower temperatures.

### Example 2

### Methanol Steam Reforming

In the typical conventional commercial methanol reforming reaction, a mixture of water and methanol at a molar concentration ratio (water:methanol) of 1.0-1.5 is pressurized to approximately 20 bar, vaporized, and heated to a temperature of 250-360° C. The resulting hydrogen is purified by purification techniques such as pressure swing adsorption. The corresponding reaction (3) is as follows:

$$\begin{matrix}{CH}_{3} OH + {H}_{2} O ⇌ {CO}_{2} + 3 {H}_{2} & {ΔH}_{r} = 49.2 kJ / mol\end{matrix} \qquad ( 3 )$$

This reaction can be carried out in a dielectric barrier discharge plasma reactor, constituted and operated in accordance with the present disclosure, at temperatures of 100° C. to 150° C. and low superatmospheric pressure, e.g., 2 bar pressure.

### Example 3

### Methane Dry Reforming

Methane dry reforming is conventionally carried out according to the following reaction (4):

$$\begin{matrix}{CH}_{4} + {CO}_{2} ⇌ 2 CO + 2 {H}_{2} & {ΔH}_{r} ( 1 , {000}^{∘} C . ) = 258.9 kJ / mol\end{matrix} \qquad ( 4 )$$

Such reaction can be carried out in a dielectric barrier discharge plasma reactor containing appropriate catalyst, in accordance with the present disclosure, at temperatures below 500° C.

### Example 4

### Ammonia Production

The Haber-Bosch process for production of ammonia was developed by German chemists Fritz Haber and Carl Bosch, in the first decade of the 20^{th} century, and converts atmospheric nitrogen (N₂) to ammonia (NH₃) by reaction with hydrogen (H₂) using a metal catalyst, under high temperatures and pressures, to carry out reaction (5).

$$\begin{matrix}{N}_{2} + 3 {H}_{2} ⇌ 2 NH 3 & {ΔH}_{r} = - 9 1.8 kJ / mol\end{matrix} \qquad ( 5 )$$

Typically, this conversion is conducted at pressure of 15-25 megapascals (MPa), (150-250 bar; 2200-3600 psi), and temperature between 400° C. and 500° C. (752° F. and 932° F.), as the gases (nitrogen and hydrogen) are passed over multiple beds of catalyst, with cooling between each pass in order to maintain an acceptable equilibrium constant. On each pass, only about 15% conversion occurs, but any unreacted gases are recycled, so that eventually an overall suitable conversion, e.g., on the order of 97%, is achieved.

Such reaction can be carried out in a dielectric barrier discharge plasma reactor containing appropriate catalyst, in accordance with the present disclosure, at ambient temperatures and pressures.

### Example 5

### Ethane Oxidative Dehydrogenation

Ethane oxidative dehydrogenation is carried out in accordance with reaction (6) below.

$$\begin{matrix}{C}_{2} {H}_{6} + {CO}_{2} ⇌ {C}_{2} {H}_{4} + CO + {H}_{2} O {ΔH}_{r} & = kJ / mol\end{matrix} \qquad ( 6 )$$

Such reaction can be carried out at substantially reduced temperature and pressure conditions with the dielectric barrier discharge plasma reactor of the present disclosure.

### Example 6

### Methane to Aromatics

The methane to aromatics reaction is carried out in accordance with reaction (7) below.

$$\begin{matrix}6 {CH}_{4} ⇌ {C}_{6} {H}_{6} + 9 {H}_{2} & {ΔH}_{r} = - kJ / mol\end{matrix} \qquad ( 7 )$$

This reaction can be carried out in the dielectric barrier discharge plasma reactor of the present disclosure at substantially reduced temperature and pressure conditions.

### Example 8

### Methane to Methanol Conversion

Methane to methanol conversion is carried out in accordance with reaction (8) below.

$$\begin{matrix}{CH}_{4} + {CO}_{2} ⇌ {CH}_{3} OH + CO & {ΔH}_{r} = - kJ / mol\end{matrix} \qquad ( 8 )$$

This reaction can be carried out in a dielectric barrier discharge plasma reactor, in accordance with the present disclosure, wherein plasma is employed to activate CH₄ and CO₂, which are very stable molecules, so that the reaction is carried out at substantially milder conditions.

It will therefore be appreciated from the foregoing description that the dielectric barrier discharge plasma reactor and method of the present disclosure are usefully employed for an extensive variety of commercially valuable reactions that heretofore have been operated at high temperatures and pressures, with substantial associated expenditures of fuel and energy to meet operational requirements for such reactions, and correspondingly substantial capital and operating costs. The apparatus and method of the present disclosure take advantage of dielectric barrier discharge plasma to activate molecular species for achievement of reaction at lower temperature and pressure conditions. Such lower temperature and pressure conditions, in turn, mean that less energy is required to be consumed by the chemical reaction process, hence achieving lower CO₂ emissions, higher efficiency, and lower costs.

The dielectric barrier discharge plasma reactor of the present disclosure entails a highly compact reactor conformation, enabling a multiplicity of such reactors to be arranged in a correspondingly sized reactor vessel, so that the reaction system may be readily scaled by the provision of greater or lesser numbers of the dielectric barrier discharge plasma reactors, as appropriate to the specific volumetric flows and requirements of the reaction system in particular applications.

While the disclosure has been set forth herein in reference to specific aspects, features and illustrative embodiments, it will be appreciated that the utility of the disclosure is not thus limited, but rather extends to and encompasses numerous other variations, modifications and alternative embodiments, as will suggest themselves to those of ordinary skill in the field of the present disclosure, based on the description herein. Correspondingly, the disclosure as hereinafter claimed is intended to be broadly construed and interpreted, as including all such variations, modifications and alternative embodiments, within its spirit and scope.

What is claimed is:

1. A dielectric barrier discharge plasma reactor, comprising
a tubular outer ground electrode having an inner surface bounding an interior volume therein;
a dielectric electrode coaxially mounted in the interior volume of the tubular outer ground electrode, the dielectric electrode comprising a central electrode in a cylindrical dielectric element, the cylindrical dielectric element having an outer surface in spaced relationship to the inner surface of the tubular outer ground electrode to define an annular fluid flow passage therebetween; and
a catalyst material comprising catalyst coated on the inner surface of the tubular outer ground electrode and optionally further comprising catalyst in a catalyst bed in the annular fluid flow passage.

2. The dielectric barrier discharge plasma reactor of claim 1, wherein the catalyst material further comprises catalyst in a catalyst bed in the annular fluid flow passage.

3. The dielectric barrier discharge plasma reactor of claim 1, wherein the cylindrical dielectric element comprises at least one dielectric material of glass, ceramic, dielectric polymers, and dielectric metal oxides.

4. The dielectric barrier discharge plasma reactor of claim 1, wherein the cylindrical dielectric element comprises quartz glass.

5. The dielectric barrier discharge plasma reactor of claim 1, wherein the cylindrical dielectric element of the dielectric electrode comprises a tube, in which the central electrode is disposed.

6. The dielectric barrier discharge plasma reactor of claim 1, wherein the cylindrical dielectric element of the dielectric electrode comprises a dielectric enamel on the central electrode.

7. A multi-tube dielectric barrier discharge plasma reactor, comprising multiple ones of the dielectric barrier discharge plasma reactor of claim 1, arranged for concurrent passage of fluid therethrough.

8. The multi-tube dielectric barrier discharge plasma reactor of claim 7, wherein the multiple ones of the dielectric barrier discharge plasma reactor are mounted in a shell.

9. The multi-tube dielectric barrier discharge plasma reactor of claim 7, wherein the multiple ones of the dielectric barrier discharge plasma reactor are parallelly aligned with one another in the shell.

10. A method of reacting fluid reactants to form reaction product(s), comprising:
flowing the fluid reactants through an annular flow passage bounded by an outer tubular ground electrode and an inner dielectric electrode in the presence of catalyst material comprising catalyst coated on the inner surface of the tubular outer ground electrode optionally further comprising catalyst in a catalyst bed in the annular fluid flow passage;
energizing the inner dielectric electrode to generate a dielectric barrier discharge plasma of the fluid reactants in the annular flow passage inducing reaction of the fluid reactants to form the reaction product(s); and
discharging the reaction products from the annular flow passage.

11. The method of claim 10, wherein the reaction comprises methane steam reforming.

12. The method of claim 10, wherein the reaction comprises methanol steam reforming.

13. The method of claim 10, wherein the reaction comprises methane dry reforming.

14. The method of claim 10, wherein the reaction comprises ammonia production.

15. The method of claim 10, wherein the reaction comprises ethane oxidative dehydrogenation.

16. The method of claim 10, wherein the reaction comprises conversion of methane to aromatics.

17. The method of claim 10, wherein the reaction comprises conversion of methane to methanol.

18. The method of claim 10, wherein the reaction comprises production of syngas from natural gas.

19. The method of claim 10, wherein the reaction comprises conversion of methane to carbon and hydrogen.

20. The method of claim 10, wherein the fluid reactants comprise methane.

21. The method of claim 10, wherein the fluid reactants comprise carbon dioxide.

22. The method of claim 10, wherein the fluid reactants comprise nitrogen.

23. The method of claim 10, wherein the energizing of the inner dielectric electrode imposes a voltage of at least 10 kV.

24. The method of claim 10, wherein the energizing of the inner dielectric electrode imposes a voltage in a range of from 10 kV to 50 kV.

25. The method of claim 10, wherein said flowing, energizing, and discharging are conducted in multiple ones of said annular flow passage, in a multi-tube dielectric barrier discharge plasma reactor.
